(19) United States
(12) Patent Application Publication
(10) Pub. No.: US 2016/0041142 A1
(43) Pub. Date: Feb. 11, 2016

(54) METHOD OF DETERMINING AND UTILIZING SCALE AND SHAPE FACTOR EQUATION COEFFICIENTS FOR RESERVOIR FLUIDS

(71) Applicants: Christopher J. Freitas, San Antonio, TX (US); Nicholas J. Mueschke, San Antonio, TX (US); Nathan F. Andrews, San Antonio, TX (US)

(72) Inventors: Christopher J. Freitas, San Antonio, TX (US); Nicholas J. Mueschke, San Antonio, TX (US); Nathan F. Andrews, San Antonio, TX (US)

(73) Assignee: Baker Hughes Incorporated, Houston, TX (US)

(21) Appl. No.: 14/451,614

(22) Filed: Aug. 5, 2014

(51) Int. Cl. G01N 33/28; G01L 11/00; G01N 25/00

(52) U.S. Cl. CPC G01N 33/2823; G01N 25/00; G01L 11/00

(57) ABSTRACT

An apparatus for estimating conditions of reservoir fluid in an underground reservoir that includes a sensor for measuring one or more measured parameters of that fluid, the measured parameters including at least one of: temperature, pressure and density of the fluid and a processor. The processor is configured to: receive data representing the one or more measured parameters; determine or receive coefficients for an extended corresponding states (XCS) model, wherein propane is used as a reference fluid in determining the coefficients or was used in the forming of the received coefficients; and solve the XCS model with the coefficients to form estimates of the fluid conditions.

### TECHNICAL FIELD

This invention relates generally to methods of calculating fluid properties and, in particular to methods to calculating fluid properties, including density, viscosity, thermal conductivity, and other thermodynamic properties for fluids in a petroleum reservoir.

### BACKGROUND

The phase behavior and physical properties of petroleum reservoir (“reservoir” hereinafter) fluids are dependent on temperature, pressure, and composition. In particular, density, viscosity, vapor-liquid equilibrium, thermal conductivity and thermodynamic derivative properties such as specific heat, enthalpy, entropy, internal energy, Joule-Thompson coefficient, and sound speed are properties of interest for reservoir fluid analyses. These properties can be required for both relatively simple fluids and complex mixtures.

A relatively simple fluid is one in which many of its defining characteristics are known and there is an abundance of experimental data, such as the single component systems of pure methane or pure ethane. A complex fluid on the other hand can be made of hundreds of components. Only small amounts of data are typically available for these types of fluids. The fluid properties for both simple and complex fluids are frequently calculated and predicted by means of numerical methods. There are many methods available in the industry and the literature, including: correlation based approaches, equations of state (EOS) models, corresponding states methodologies, and many other unique approaches.

Correlation approaches are very prevalent in the literature and industry and exist for many important reservoir fluid properties including density, viscosity, thermal conductivity, specific heats, etc. Correlations are generally straight-forward to implement and can provide very accurate results compared to experimental data. However, the predictive capability of correlations can be limited to experimental data ranges and often do not extrapolate well beyond these ranges. Furthermore when properties of uncommon or complex multi-component fluids are desired, there may not be adequate data sets or correlations to describe the fluid properties. Therefore, more robust methods are often required for application.

Equations of state (EOS) are an attractive alternative to correlations for calculating fluid properties due to their ability to provide reliable calculations, be generalized to single or multi-components systems, and to be used in a predictive manner. They can however be more involved to implement, limited in applicability, and suffer in accuracy, especially with increasing generality. For example, highly accurate fluid specific EOS models such as the 32 term Benedict-Webb-Rubin (BWR) EOS model have been developed to accurately represent fluid properties, however they are limited because they are only developed uniquely for fluids that have a wealth of experimental data. Other types of EOS models have been developed to target generality, including the cubic EOS models which are the most common and successful type in the reservoir engineering industry. While these methods provide accurate predictive capabilities for vapor-liquid fluid equilibrium and vapor properties, they have also been found to be unreliable and inaccurate for calculating liquid densities and thermodynamic derivative properties.

To address the shortcomings of cubic EOS model, many have modified cubic EOS model formulations, including modifying mixing rules and other parameter equations. For example, methodologies such as volume shift techniques have been created to improve predictions of liquid density values.

Other methods have been researched to calculate the thermodynamic properties of reservoir fluids. Such methods include the application of different mixing rules, cubic plus association (CPA) models, and statistical association models (such as statistical association fluid theory (SAFT)). These models, however, have yet to be widely successful and generally accepted for reservoir engineering applications. Corresponding states models are yet one more alternative to calculate fluid properties. These have seen some success in reservoir engineering applications.

In particular, extended corresponding states (XCS) models have been developed that are useful in computing both thermodynamic (properties such as density, thermodynamic derivative properties) and transport properties (such as viscosity and thermal conductivity). The basic theory of corresponding states takes a pure fluid with properties that can be calculated with a high degree of accuracy as a reference fluid, and then applies scaling arguments to calculate properties for a fluid of interest. Application of these scaling arguments to governing equations results in parameters that are typically referred to as scale factors. When used with non-conformal fluids, scale factors can be functions of additional factors termed molecular shape factors. The application of these molecular shape factors is what is referred to as extended corresponding states and serves to broaden the range of applicability of the corresponding states method.

XCS models may have the advantage of being theoretically based and predictive, rather than just correlative and empirical. Thus, a sacrifice in accuracy in comparison to a well-developed correlation or EOS model is traded for generality and predictive power outside of experimental data ranges.

Typically there are two shape factors in the XCS formulation that affect the calculation of all properties; one applied to a temperature scale factor and another applied to a density scale factor. A key part of any XCS method is the means to calculate these shape factors. There are many documented methods for doing this in the open literature. The simplest form is to use constant values determined a priori. For more accurate shape factors, available experimental pressure-volume-temperature (PVT) data or other accurate property data for a fluid of interest may be fit to an XCS scale factor formulation and generate “exact” scale factors at any point in the valid PVT space. These “exact” scale factors can then be used to create shape factor correlations as functions of temperature and/or density. As an alternative to using PVT data across a full PVT space, for fluids with minimal data, saturation boundary data and/or accurate equations can be used to generate shape factor correlation equations. Unique correlations can be created on a per fluid basis using a single fluid's data set, or generalized correlations can be developed by fitting scale factor data from multiple fluids of interest simultaneously. These correlations are one approach to extrapolate properties outside of the available data range and to calculate the properties of similar type fluids without the need for abundant experimental data.

### SUMMARY

According to one embodiment, an apparatus for estimating conditions of reservoir fluid in an underground reservoir that includes a sensor for measuring one or more measured parameters of that fluid, the measured parameters including at least one of: temperature, pressure and density of the fluid and a processor is disclosed. The processor is configured to: receive data representing the one or more measured parameters; determine or receive coefficients for an extended corresponding states (XCS) model, wherein propane is used as a reference fluid in determining the coefficients or was used in the forming of the received coefficients; and solve the XCS model with the coefficients to form estimates of the fluid conditions.

According to another embodiment, a computer based method estimating conditions of reservoir fluid in an underground reservoir is disclosed and includes determining coefficients for an extended corresponding states (XCS) model. The determination includes: calculating saturation pressure of the component of interest; forming an initial estimate of a first scale factor; forming a propane equivalent temperature based on the initial estimate of the first scale factor and a measured temperature; iteratively revising the initial estimate until convergence is reached to form a first scale factor; calculating a second scale factor; and regressing the first and second scale factors. The method also includes solving the XCS model with the coefficients to form estimates of the fluid conditions.

Also disclosed is a computer based method of estimating conditions of reservoir fluid in an underground reservoir that includes receiving coefficients for an extended corresponding states (XCS) model, wherein propane was used as a reference fluid in forming the received coefficients and first and second scale factors in the coefficients were regressed over a range of temperatures.

### BRIEF DESCRIPTION OF THE DRAWINGS

The subject matter, which is regarded as the invention, is particularly pointed out and distinctly claimed in the claims at the conclusion of the specification. The foregoing and other features and advantages of the invention are apparent from the following detailed description taken in conjunction with the accompanying drawings, wherein like elements are numbered alike, in which:

FIG. 1 shows an example drilling system according to one embodiment;

FIG. 2 is a flow diagram of method according to one embodiment; and

FIG. 3 is a flow diagram of method according to another embodiment.

### DETAILED DESCRIPTION

Embodiments disclosed herein may provide clear, straightforward, and reliable method for generating and using shape factor correlations for use in reservoir fluid property predictions by means of XCS methodologies.

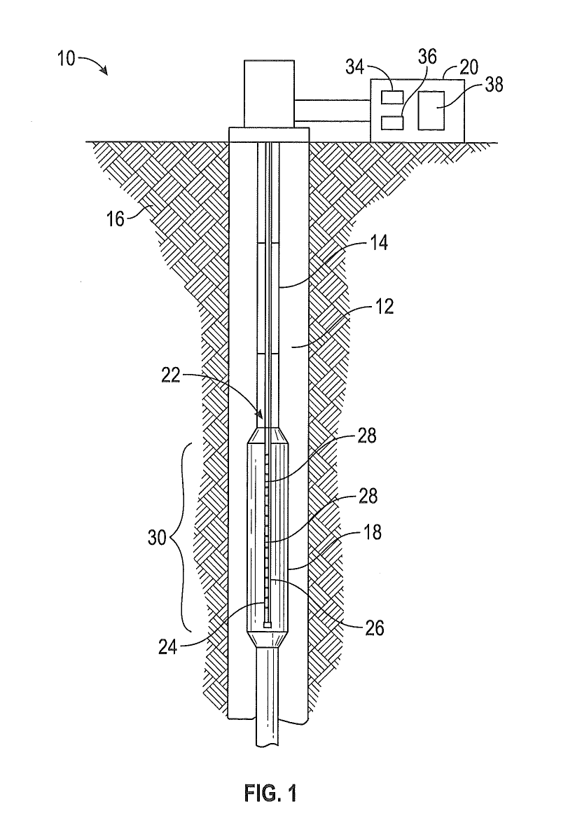

Referring to FIG. 1, an exemplary embodiment of a downhole drilling, monitoring, evaluation, exploration and/or production system 10 disposed in a wellbore 12 is shown. A borehole string 14 is disposed in the wellbore 12, which penetrates at least one earth formation 16 for performing functions such as extracting matter from the formation and/or making measurements of properties of the formation 16 and/or the wellbore 12 downhole. The borehole string 14 is made from, for example, a pipe, multiple pipe sections or flexible tubing. The system 10 and/or the borehole string 14 include any number of downhole tools 18 for various processes including drilling, hydrocarbon production, and measuring one or more physical quantities in or around a borehole. Various measurement tools 18 may be incorporated into the system 10 to affect measurement regimes such as wireline measurement applications or logging-while-drilling (LWD) applications.

In one embodiment, a parameter measurement system is included as part of the system 10 and is configured to measure or estimate various downhole parameters of the formation 16, the borehole 14, the tool 18 and/or other downhole components. The illustrated measurement system includes an optical interrogator or measurement unit 20 connected in operable communication with at least one optical fiber sensing assembly 22. The measurement unit 20 may be located, for example, at a surface location, a subsea location and/or a surface location on a marine well platform or a marine craft. The measurement unit 20 may also be incorporated with the borehole string 12 or tool 18, or otherwise disposed downhole as desired.

In the illustrated embodiment, an optical fiber assembly 22 is operably connected to the measurement unit 20 and is configured to be disposed downhole. The optical fiber assembly 22 includes at least one optical fiber core 24 (referred to as a “sensor core” 24) configured to take a distributed measurement of a downhole parameter (e.g., temperature, pressure, stress, strain and others). In one embodiment, the system may optionally include at least one optical fiber core 26 (referred to as a “system reference core” 26) configured to generate a reference signal. The sensor core 24 includes one or more sensing locations 28 disposed along a length of the sensor core, which are configured to reflect and/or scatter optical interrogation signals transmitted by the measurement unit 20. Examples of sensing locations 28 include fibre Bragg gratings, Fabry-Perot cavities, partially reflecting mirrors, and locations of intrinsic scattering such as Rayleigh scattering, Brillouin scattering and Raman scattering locations. If included, the system reference core 26 may be disposed in a fixed relationship to the sensor core 24 and provides a reference optical path having an effective cavity length that is stable relative to the optical path cavity length of the sensor core 24.

In one embodiment, a length of the optical fiber assembly 22 defines a measurement region 30 along which distributed parameter measurements may be taken. For example, the measurement region 30 extends along a length of the assembly that includes sensor core sensing locations 28.

The measurement unit 20 includes, for example, one or more electromagnetic signal sources 34 such as a tunable light source, a LED and/or a laser, and one or more signal detectors 36 (e.g., photodiodes). Signal processing electronics may also be included in the measurement unit 20, for combining reflected signals and/or processing the signals. In one embodiment, a processing unit 38 is in operable communication with the signal source 34 and the detector 36 and is configured to control the source 34, receive reflected signal data from the detector 36 and/or process reflected signal data.

In one embodiment, the measurement system is configured as a coherent optical frequency-domain reflectometry (OFDR) system. In this embodiment, the source 34 includes a continuously tunable laser that is used to spectrally interrogate the optical fiber sensing assembly 22.

The optical fiber assembly 22 and/or the measurement system are not limited to the embodiments described herein, and may be disposed with any suitable carrier. That is, while an optical fiber assembly 22 is shown, any type of now known or later developed manners of obtaining information relative a reservoir may be utilized to measure various information (e.g., temperature, pressure, salinity and the like) about fluids in a reservoir. Thus, in one embodiment, the measurement system may not employ any fibers at all and may communicate data electrically.

A “carrier” as described herein means any device, device component, combination of devices, media and/or member that may be used to convey, house, support or otherwise facilitate the use of another device, device component, combination of devices, media and/or member. Exemplary non-limiting carriers include drill strings of the coiled tube type, of the jointed pipe type and any combination or portion thereof. Other carrier examples include casing pipes, wirelines, wireline sondes, slickline sondes, drop shots, downhole subs, bottom-hole assemblies, and drill strings.

In support of the teachings herein, various analysis components may be used, including a digital and/or an analog system. Components of the system, such as the measurement unit 20, the processor 38, the processing assembly 50 and other components of the system 10, may have components such as a processor, storage media, memory, input, output, communications link, user interfaces, software programs, signal processors (digital or analog) and other such components (such as resistors, capacitors, inductors and others) to provide for operation and analyses of the apparatus and methods disclosed herein in any of several manners well appreciated in the art. It is considered that these teachings may be, but need not be, implemented in conjunction with a set of computer executable instructions stored on a computer readable medium, including memory (ROMs, RAMs), optical (CD-ROMs), or magnetic (disks, hard drives), or any other type that when executed causes a computer to implement the method of the present invention. These instructions may provide for equipment operation, control, data collection and analysis and other functions deemed relevant by a system designer, owner, user or other such personnel, in addition to the functions described in this disclosure.

Further, various other components may be included and called upon for providing for aspects of the teachings herein. For example, a power supply (e.g., at least one of a generator, a remote supply and a battery), cooling unit, heating unit, motive force (such as a translational force, propulsional force or a rotational force), magnet, electromagnet, sensor, electrode, transmitter, receiver, transceiver, antenna, controller, optical unit, electrical unit or electromechanical unit may be included in support of the various aspects discussed herein or in support of other functions beyond this disclosure.

The saturation boundary technique for generating scale and shape factors coefficients disclosed herein utilizes pressure, temperature, and density data along the vapor-liquid saturation curve. This data is required for both pure component fluids of interest and a reference fluid. Specifically, the data is used to derive highly accurate values for scale factors along the saturation curve. Once these scale factors are generated, shape factor correlation equations can be fit to the scale factor curves. If the chosen shape factor correlations are chosen appropriately, this allows shape factors to be smoothly extrapolated and calculated outside of the saturation conditions. Thus, property predictions can be made at a broad range of thermodynamic states.

Researchers have used the saturation boundary technique, along with the full PVT space “exact” shape factor method described above, to generate shape factor correlations for reservoir fluids. This research suggests that the density shape factor correlation is calculated first and then the temperature shape factor is generated leveraging the density shape factor correlation equation with the saturation data. Others have stated that the saturation boundary method can be used with reservoir fluids.

At present, there are no openly available component specific shape factor equation coefficients for hydrocarbon reservoir fluids. In addition, the methods used in previous works to find coefficients are ambiguous in the actual process used and the saturation data employed to create the equations.

One or more embodiments disclosed herein may calculate the shape factor correlation equation coefficients. In one embodiment, a set of industry accepted and openly available equations are utilized in a different manner to generate a new effective shape factor calculation routine. In one embodiment, a method for generating shape factor correlations for use in reservoir fluid XCS property predictions that may be applied to most reservoir fluids is disclosed.

By way a further background a foundation of necessary details is provided for the formulation of XCS is provided and is mostly found in the works of Huber and Hanley with differences and additions noted as applicable.

### XCS Formulation

XCS models establish that two conformal or non-conformal fluids (non-conformal meaning that the reduced intermolecular potentials are not equal) can be related by the scaling law:

aⱼ^{r}(ρⱼ,Tⱼ)=aⱼ^{r}(ρₒ,Tₒ) (1)

where a^{r} is the reduced residual Helmholtz free energy, ρ is the density, T is temperature, the subscript j refers to the component index in the fluid of interest, and the subscript o is the reference fluid.

The scaling arguments which support equation (1) are

Tₒ=Tⱼ/fⱼ (2); and

ρₒ=ρⱼhⱼ (3)

where f is the temperature scale factor and h is density scale factor. The scale factors are formally functions of temperature and density, however in this work they are assumed independent of density. This assumption yields an equivalent compressibility factor relationship, namely Zⱼ=Zₒ. Therefore pressure, P, can be expressed as Pₒ=Pⱼ(hⱼ/fⱼ). Herein, scale factors are calculated with one of two methods: (1) the saturation boundary scale factor method when the temperature and pressure of interest lies within the saturation boundary range of the fluid or (2) as functions of shape factor correlation equations at other temperature and pressure conditions. The scale factors can be defined as functions of shape factors by the relations:

$$\begin{matrix}{f}_{j} = \frac{{T}_{j}^{c}}{{T}_{o}^{c}} {θ}_{j} ( ρ , T ) ; and & ( 4 ) \\ {h}_{j} = \frac{{ρ}_{o}^{c}}{{ρ}_{j}^{c}} {φ}_{j} ( ρ , T ) & ( 5 )\end{matrix}$$

where the superscript c is the value at critical conditions and θ and φ are the temperature and density shape factors, respectively. The shape factors are shown formally here as functions of temperature and density, but density may be omitted in some instances. The two shape factors are found herein by means of saturation boundary extrapolation correlations as explained later.

For fluid mixtures, the van der Waals mixing rules can be applied as follows:

$$\begin{matrix}{h}_{x} = {\sum }_{i = 1}^{n} {\sum }_{j = 1}^{n} {x}_{i} {x}_{j} {h}_{ij} ; and & ( 6 ) \\ {f}_{x} {h}_{x} = {\sum }_{i = 1}^{n} {\sum }_{j = 1}^{n} {x}_{i} {x}_{j} {f}_{ij} {h}_{ij} & ( 7 )\end{matrix}$$

with cross terms equal to

fᵢⱼ=\sqrt{fᵢfⱼ}(1−kᵢⱼ) (8); and

hᵢⱼ=(hᵢ^{1/3}+hⱼ^{1/3})³(1−lᵢⱼ)/8 (9)

where n is the number of components in the mixture, x is the mole fraction of the component in the mixture, and kᵢⱼ and lᵢⱼ are the binary interaction parameters. kᵢⱼ is assumed zero for hydrocarbon/hydrocarbon pairs and the values from Pedersen and Christensen are assumed for pairs with carbon dioxide, nitrogen and hydrogen sulfide. All values for lᵢⱼ are assumed equal to zero.

With these equations, the density can be readily solved once values for the scale and shape factors are obtained. In addition, derivative thermodynamic properties can be calculated by differentiating equation (1). The details of this procedure are available in the literature and are not repeated here.

For the transport properties of viscosity and thermal conductivity, no direct relationship is known as a consequence of equation (1). Instead the properties themselves are also related by means of an extended corresponding states relation.

For viscosity, the property of the fluid of interest can be obtained:

nⱼ(ρⱼ,Tⱼ)=n*ⱼ(Tⱼ)+[ηₒ(ρₒ,Tₒ)−n*ₒ(Tₒ)]F_{η}+Δη_{Enskog}(ρⱼ) (10)

where η is viscosity, η* is the dilute viscosity as calculated by the Lennard-Jones force law, Δη_{Enskog} is a correction to viscosity, and F_{η} is a viscosity adjustment factor defined by:

F_{η}=fₓ^{1/2}hₓ^{-2/3}gₓ^{1/2}. (11)

Here f and h are obtained from the equations above and g is either a mass scale factor or a saturation viscosity scale factor. The mass scale factor is defined as:

$$\begin{matrix}{g}_{x , η}^{1 / 2} = {M}_{o}^{- 1 / 2} {f}_{x}^{- 1 / 2} {h}_{x}^{- 4 / 3} [ {\sum }_{i = 1}^{n} {\sum }_{j = 1}^{n} {x}_{i} {x}_{j} {M}_{ij , η}^{1 / 2} {f}_{ij}^{1 / 2} {h}_{ij}^{4 / 3} ] ; and & ( 12 ) \\ {M}_{ij , η} = ( 2 {M}_{i} {M}_{j} ) / ( {M}_{i} + {M}_{j} ) & ( 13 )\end{matrix}$$

where M is the molecular weight. When saturation viscosity data is available, g is a saturation viscosity scale factor defined as:

$$\begin{matrix}{g}_{x , η}^{1 / 2} = \frac{{η}_{i , saturated} ( {ρ}_{i} , {T}_{o} / {f}_{i} ) - {η}_{i}^{*} ( {T}_{o} / {f}_{i} )}{[ {{η}_{o , saturated} ( {ρ}_{o} , {T}_{o} )}_{i} - {{η}_{o}^{*} ( {T}_{o} )}_{i} ] {f}_{i}^{1 / 2} {h}_{i}^{- 2 / 3}} , & ( 14 ) ; \\ {g}_{ij , η} = \frac{2}{( 1 / {g}_{i} + 1 / {g}_{j} )} , and & ( 15 ) ; \\ {g}_{x , η}^{1 / 2} {f}_{x}^{1 / 2} {h}_{x}^{4 / 3} = [ {\sum }_{i = 1}^{n} {\sum }_{j = 1}^{n} {x}_{i} {x}_{j} {g}_{ij , η}^{1 / 2} {f}_{ij}^{1 / 2} {h}_{ij}^{4 / 3} ] . & ( 16 )\end{matrix}$$

In tests of the method disclosed herein propane was used as the reference fluid.

### Saturation Boundary Method for Reservoir Fluids

The scale factors of a fluid of interest can be calculated with respect to the reference fluid by simultaneously solving the saturation equations:

Pⱼ^{sat}(T)=Pₒ^{sat}(T/fⱼ)fⱼ/hⱼ (17); and

ρⱼ^{sat}(T)=ρₒ^{sat}(T/fⱼ)/hⱼ (18).

Solving these equations require saturation data which can be acquired by either experimental data or correlations. With these values, the temperature and density scale factors can be readily solved with an iterative method to generate scale factors. Once these scale factors are calculated in the saturation ranges, they can be used in the XCS calculations. However, outside of the saturation conditions, the scale factors must be determined a different way. The common way is to extrapolate the saturation scale factors to temperatures and pressures outside of the saturation range.

Extrapolation is enabled when the shape factors of each scale factor for the saturation region are cast into a correlation model form for use in the scale factor equations (4) and (5). Constants of the predetermined shape factor correlation equations may be expressed as:

θᵢ=1+(ωᵢ−ωₒ)(α₁+α₂ ln Tᵣ) (19); and

φᵢ=(Zₒ^{c}/Zᵢ^{c})[1−(ωᵢ−ωₒ)(β₁+β₂ ln Tᵣ)] (20).

where Z^{c} is the critical compressibility factor, ω is the acentric factor, Tᵣ is the reduced temperature (equal to T/T^{c}), and α₁, α₂, β₁ and β₂ are coefficients determined in the regression process. The selected model forms are only temperature dependent (i.e., independent of density) and have smooth extrapolation properties.

One difference between the above and embodiments disclosed herein relate to the formulations used in the XCS foundation, and the different use of saturation equations for the fluids. For the saturation data, as opposed to independently fitting data to the saturation equation, the works of others that have been previously done and are openly available in the literature may be leveraged. This provides easy access and ease of duplication of the work. For the saturation pressure of the fluids of interest, the modified Wagner equation from the API-TDB (American Petroleum Institute—Technical Data Book) as reported in Riazi is employed:

$$ln ( {P}_{r}^{vap} ) = \frac{a τ + b {τ}^{1.5} c {τ}^{2.6} + d {τ}^{5}}{{T}_{r}} \qquad ( 21 )$$

where τ=1−Tᵣ, Pᵣ is the reduced pressure defined as Pᵣ=P/P_{c} and a-d are material specific constants for this equation. The liquid saturation density values of the fluids of interest may be calculated with the correlation:

$$\frac{{V}_{s}}{{V}^{o}} = {V}_{R}^{( 0 )} ( 1 - {ω}_{SRK} {V}_{R}^{( δ )} ) \\ {V}_{R}^{( 0 )} = \begin{matrix}1 - 1.52816 {( 1 - {T}_{R} )}^{1 / 3} + 1.43907 {( 1 - {T}_{R} )}^{2 / 3} - \\ 0.81446 ( 1 - {T}_{R} ) - 0.296123 {( 1 - {T}_{R} )}^{4 / 3}\end{matrix} \\ {V}_{R}^{( δ )} = ( \begin{matrix}- 0.296123 + 0.386914 {T}_{R} + \\ - 0.0427258 {T}_{R}^{2} + - 0.0480648 {T}_{R}^{2}\end{matrix} ) / ( {T}_{R} - 1.00001 ) \\ 0.25 < {T}_{R} < 0.95 \qquad ( 22 )$$

where V is volume, V^{o} is the characteristic volume, and ω_{SRK} is the acentric factor for the Soave-Redlich-Kwong (SRK) equation of state. V^{o} and ω_{SRK} are material specific constants for this equation and are specified by Hankinson and Thomson. With these values, scale factor curves were generated along the saturation boundary. This was done in the range of 0.35<Tr<0.90 or the range as specified by the validated limits of the vapor pressure equation, whichever was the most restrictive. Both scale factors are calculated simultaneously from equations (19) and (20) as a pair. Subsequently, both f and h are then used to calculate the shape factors from equations (4) and (5) that could be regressed against the shape factor correlation forms independently of each other. This is different than prior methods where the value for θ was first cast into correlation form and then used in a second scale factor generation sequence to create the h curves before regression to the shape factor equation for φ.

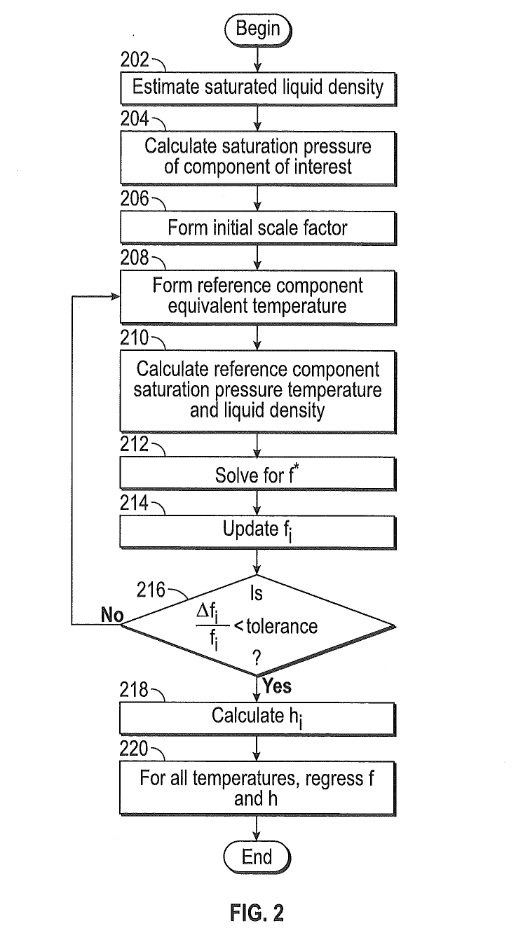

In more detail, scale and shape factors of individual pure components for use in an extended corresponding states (XCS) property model may be generated based on the method shown in FIG. 2. At block 202 a saturated liquid density of the component of interest may be calculated. In one embodiment, this calculation includes utilizing equation (22) above. The estimate may include using values of temperature and pressure measured in the reservoir.

At block 204 the saturation pressure of the pure component may be calculated with the known API-TDB equation, equation 21 above.

At block 206 an initial guess of the first scale factor (f) may be generated. This initial guess may utilize, for example the following equation:

$${f}_{I} = ( {T}_{c , i} / {T}_{c , 0} ) {θ}_{i}$$

$${θ}_{i} = 1 + ( {ω}_{i} - {ω}_{0} ) ( {α}_{1} + {α}_{2} ln ( {T}_{r , i} ) )$$

where: ∝₁=0.06354, and ∝₂=0.7256, ωᵢ is the acentric factor of the component of interest and ωₒ is the acentric factor of the reference fluid

At block 208 the propane equivalent temperature is calculated:

$${T}_{i , o} = \frac{T}{{f}_{i}} .$$

At block 210 the saturation pressure and liquid density of propane at the equivalent propane temperature is calculated, with the following equations applied at the equivalent temperature for the propane saturation pressures:

$$ln ( {P}_{sat , o} ) = \begin{matrix}ln ( {P}_{t , o} ) + {V}_{p , 1} x + {V}_{p , 2} {x}^{2} + \\ {V}_{p , 3} {x}^{3} + {V}_{p , 4} {x}^{4} + {V}_{p , 5} {x ( 1 - x )}^{{V}_{p , 6}}\end{matrix}$$

$${V}_{p , 1} = 15.410153272$$

$${V}_{p , 2} = 11.870733615$$

$${V}_{p , 3} = - 0.874958355$$

$${V}_{p , 4} = - 2.448971934$$

$${V}_{p , 5} = 11.400962259$$

$${V}_{p , 6} = 1.2$$

$${P}_{t} = 1.6850 \times {10}^{- 10}$$

$$x = \frac{1 - 369.85 / T}{1 - 85.47 / 369.85}$$

and for the propane saturation density:

$${ρ}_{sat , L , o} = {ρ}_{c , o} + ( 16636 - 5000 ) exp ( v ( t ) )$$

$$v ( t ) = A ( 7 ) ln x + {\sum }_{n = 8}^{10} A ( n ) ( 1 - {x}^{( n - 11 ) / 3 )} ) + {\sum }_{n = 11}^{13} A ( n ) ( 1 - {x}^{( n - 10 ) / 3} )$$

$$x = \frac{369.85 - T}{369.85 - 85.47}$$

$$A ( 1 ) = 0.277609660772$$

$$A ( 2 ) = 0.0996316211526$$

$$A ( 3 ) = - 0.0935103011479$$

$$A ( 4 ) = - 0.93181193381$$

$$A ( 5 ) = 0.78039332334$$

$$A ( 6 ) = - 0.594672655236$$

$$A ( 7 ) = - 17.0353717858$$

$$A ( 8 ) = 0.0850718580945$$

$$A ( 9 ) = - 1.69899508271$$

$$A ( 10 ) = 18.4206833899$$

$$A ( 11 ) = - 81.5334435591$$

$$A ( 12 ) = 33.0612340278$$

$$A ( 13 ) = - 7.37636511031$$

as specified by Younglove and Ely.

The convergence of these estimates may then be checked by first solving for the Newton-Raphson iteration variable f* at block 212. This may include solving:

$${f}^{*} = \frac{{P}_{vp} {ρ}_{sat , 0}}{{P}_{vp , 0} {ρ}_{sat}} - {f}_{i}$$

At block 214 fᵢ is updated. This may include solving:

$${f}_{i} = {f}_{i} - \frac{{f}^{*}}{\frac{\partial {f}^{*}}{\partial {f}_{i}}}$$

At block 216 is it determined if convergence has occurred. This may include determining whether:

$$\frac{Δ {f}_{i}}{{f}_{i}} < tolerance$$

If not, the processing in blocks 208-214 is repeated. If it is, processing begins in block 218 where the second scale factor (h) is calculated. In one embodiment this may include solving:

$${h}_{i} = \frac{{ρ}_{sat , 0}}{{ρ}_{sat}} .$$

At block 220, for all temperatures, the scale factors f and h are regressed according to the following equations to determine the α and β terms in the temperature and density shape factor correlations:

$${f}_{I} = ( \frac{{T}_{c , i}}{{T}_{c , 0}} ) {θ}_{i} and$$

$${h}_{1} = ( \frac{{ρ}_{c , 0}}{{ρ}_{c , j}} ) {φ}_{i} ;$$

$$Where {θ}_{i} = 1 + ( {ω}_{i} - {ω}_{0} ) ( {α}_{1} + {α}_{2} ln ( {T}_{r , i} ) ) and$$

$${φ}_{i} = ( \frac{{z}_{c , 0}}{{z}_{c , i}} ) [ 1 - ( {ω}_{i} - {ω}_{0} ) ( {β}_{1} + {β}_{2} ln ( {T}_{r , i} ) ) ] .$$

An example of the results of the performing the method shown in FIG. 2 is illustrated by table 1 below:

| | Tmin | Tmax | | | | | | R-squared, | ARE, | R-squared, | ARE, |
|---|---|---|---|---|---|---|---|---|---|---|---|
| | (K) | (K) | Equations | α₁ | α₂ | β₁ | β₂ | α | α | β | β |
| C1 | 91.0 | 181.0 | (19)-(20) | 0.06595530 | −0.74654082 | 0.22237743 | −0.21709153 | 0.97631 | −6.87E−07 | 0.97749 | −6.26E−07 |
| C2 | 106.9 | 290.1 | (19)-(20) | 0.06864320 | −0.74624710 | 0.16343000 | −0.21530066 | 0.99864 | −1.53E−07 | 0.98591 | −1.24E−07 |
| nC4 | 148.8 | 403.9 | (19)-(20) | 0.08974334 | −0.61532270 | 0.22553000 | −0.13237654 | 0.99501 | 5.54E−04 | 0.91654 | 5.10E−04 |
| iC4 | 142.9 | 387.7 | (19)-(20) | −0.03489384 | −0.86807330 | 0.03561000 | 0.00436264 | 0.97394 | −1.28E−06 | 0.00339 | −3.79E−07 |
| nC5 | 164.4 | 446.3 | (19)-(20) | 0.06051728 | −0.73931460 | 0.24602000 | −0.14208699 | 0.99754 | 9.59E−04 | 0.91968 | 1.12E−03 |
| iC5 | 178.0 | 437.4 | (19)-(20) | 0.06986468 | −0.67120030 | 0.15574000 | −0.16421801 | 0.99757 | −3.32E−07 | 0.97412 | −2.37E−07 |
| 22DMC3 | 257.0 | 412.1 | (19)-(20) | −0.01013802 | −0.81012305 | −0.01034289 | 0.00808222 | 0.99989 | −2.12E−09 | 0.01525 | −1.20E−07 |
| C6 | 178.0 | 482.5 | (19)-(20) | 0.07111134 | −0.74468150 | 0.27742000 | −0.16157445 | 0.99394 | −4.23E−06 | 0.92048 | −3.79E−06 |
| C7 | 189.0 | 513.1 | (19)-(20) | 0.06281580 | −0.71988750 | 0.24737000 | −0.14951215 | 0.99909 | −1.08E−06 | 0.96347 | −2.68E−06 |
| C8 | 286.0 | 540.4 | (19)-(20) | 0.06799288 | −0.70416496 | 0.22970724 | −0.19544079 | 0.99874 | −8.40E−07 | 0.99289 | −9.11E−07 |
| C9 | 233.0 | 564.8 | (19)-(20) | 0.09383912 | −0.63030280 | 0.23444000 | −0.18552420 | 0.99546 | −6.45E−06 | 0.96791 | −6.58E−06 |
| C10 | 286.0 | 586.8 | (19)-(20) | 0.06865959 | −0.69966980 | 0.22490000 | −0.18370611 | 0.99911 | −1.44E−06 | 0.98777 | −2.10E−06 |
| C11 | 322.0 | 606.8 | (19)-(20) | 0.07195859 | −0.71378250 | 0.23458000 | −0.19946499 | 0.99909 | −1.48E−06 | 0.98930 | −2.17E−06 |
| C12 | 294.0 | 625.3 | (19)-(20) | 0.07198171 | −0.70402090 | 0.22838000 | −0.18157818 | 0.99934 | −1.74E−06 | 0.98309 | −5.20E−06 |
| C13 | 333.0 | 642.3 | (19)-(20) | 0.09415536 | −0.63808268 | 0.20802658 | −0.21748250 | 0.98759 | −2.21E−05 | 0.98699 | −5.04E−06 |
| C14 | 369.0 | 658.3 | (19)-(20) | 0.07099393 | −0.72885861 | 0.22187118 | −0.20949819 | 0.99929 | −1.62E−06 | 0.98951 | −3.51E−06 |
| C15 | 393.0 | 671.4 | (19)-(20) | 0.07160255 | −0.72021820 | 0.22038000 | −0.21308442 | 0.99921 | −1.74E−06 | 0.99037 | −3.46E−06 |
| C16 | 294.0 | 684.5 | (19)-(20) | 0.07666249 | −0.69443140 | 0.23137000 | −0.17211826 | 0.99932 | −3.86E−06 | 0.97745 | −1.72E−05 |
| C17 | 311.0 | 696.7 | (19)-(20) | 0.06236234 | −0.74075040 | 0.24892000 | −0.16559151 | 0.99803 | −1.05E−05 | 0.97477 | −1.91E−05 |
| C18 | 322.0 | 708.0 | (19)-(20) | 0.07251220 | −0.70514812 | 0.24821311 | −0.16936427 | 0.99988 | −6.12E−07 | 0.97335 | −2.22E−05 |
| C19 | 400.0 | 718.2 | (19)-(20) | 0.07136466 | −0.70808990 | 0.27795000 | −0.18343332 | 0.99971 | −1.08E−06 | 0.98507 | −9.02E−06 |
| C20 | 353.0 | 728.8 | (19)-(20) | 0.06018169 | −0.76252630 | 0.27344000 | −0.16815771 | 0.99831 | −1.01E−05 | 0.99047 | −8.36E−06 |
| C24 | 447.0 | 777.1 | (19)-(20) | 0.08881223 | −0.74016190 | 0.23873000 | −0.21573849 | 0.99992 | −3.57E−07 | 0.97925 | −2.20E−05 |
| CO2 | 217.0 | 289.0 | (19)-(20) | 0.00986229 | −0.82920580 | 0.18761000 | −0.03564042 | 0.99948 | −7.89E−09 | 0.85200 | −5.18E−09 |
| N2 | 64.0 | 119.9 | (19)-(20) | 0.09881991 | −0.71028900 | 0.33958000 | −0.30215451 | 0.99767 | 6.24E−04 | 0.96878 | 9.51E−04 |
| H25 | 188.0 | 354.5 | (19)-(20) | 0.24448173 | −0.69614827 | 0.47917754 | −0.68789781 | 0.99700 | −7.44E−08 | 0.89479 | −2.56E−06 |

The coefficients shown above may be utilized in, for example, XCS property models such as those described above for density and viscosity, in addition to other thermodynamic property and thermal conductivity XCS model forms.

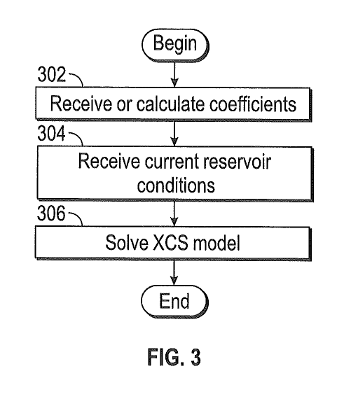

Accordingly, in one embodiment, a method as shown in FIG. 3 may be implemented where, at block 302 coefficients for a particular element are either calculated as described above or are received (e.g., observed from a table). Current reservoir conditions such as temperature, pressure and density (two of these reservoir state parameters are required)) at block 304 are also received. The coefficients are then utilized at block 306 in an XCS property model. Any or all of the above steps may be performed on a computing device.

While the invention has been described with reference to exemplary embodiments, it will be understood that various changes may be made and equivalents may be substituted for elements thereof without departing from the scope of the invention. In addition, many modifications will be appreciated to adapt a particular instrument, situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An apparatus for estimating conditions of reservoir fluid in an underground reservoir, the apparatus comprising:
a sensor for measuring one or more measured parameters of that fluid, the measured parameters including at least one of: temperature, pressure and density of the fluid; and
a processor, the processor configured to:
receive data representing the one or more measured parameters;
determine or receive coefficients for an extended corresponding states (XCS) model, wherein propane is used as a reference fluid in determining the coefficients or was used in the forming of the received coefficients; and
solve the XCS model with the coefficients to form estimates of the fluid conditions.

2. The apparatus of claim 1, wherein the processor determines the coefficients by:
estimating a saturated liquid density for a component of interest;
calculate saturation pressure of the component of interest;
form an initial estimate of a first scale factor;
form a propane equivalent temperature based on the initial estimate of the first scale factor and a measured temperature;
iteratively revising the initial estimate until convergence is reached to form a first scale factor;
calculate a second scale factor; and
regress the first and second scale factors.

3. The apparatus of claim 2, wherein the first scale factor is denoted fᵢ and the second scale factor is denoted hᵢ and wherein regressing includes solving:
$${f}_{I} = ( \frac{{T}_{c , i}}{{T}_{c , 0}} ) {θ}_{i} and$$
$${h}_{1} = ( \frac{{ρ}_{c , 0}}{{ρ}_{c , j}} ) {φ}_{i} ;$$
$$where {θ}_{i} = 1 + ( {ω}_{i} - {ω}_{0} ) ( {α}_{1} + {α}_{2} ln ( {T}_{r , i} ) ) and$$
$${φ}_{i} = ( \frac{{z}_{c , 0}}{{z}_{c , i}} ) [ 1 - ( {ω}_{i} - {ω}_{0} ) ( {β}_{1} + {β}_{2} ln ( {T}_{r , i} ) ) ] .$$

4. A computer based method estimating conditions of reservoir fluid in an underground reservoir, the method including comprising:
determining coefficients for an extended corresponding states (XCS) model, determining including:
calculating saturation pressure of the component of interest;
forming an initial estimate of a first scale factor;
forming a propane equivalent temperature based on the initial estimate of the first scale factor and a measured temperature;
iteratively revising the initial estimate until convergence is reached to form a first scale factor;
calculating a second scale factor; and
regressing the first and second scale factors; and
solving the XCS model with the coefficients to form estimates of the fluid conditions.

5. A computer based method of estimating conditions of reservoir fluid in an underground reservoir, the method including comprising:
receiving coefficients for an extended corresponding states (XCS) model, wherein propane was used as a reference fluid in forming the received coefficients and first and second scale factors in the coefficients were regressed over a range of temperatures.
